(19) United States
(12) Patent Application Publication
(10) Pub. No.: US 2024/0325184 A1
(43) Pub. Date: Oct. 3, 2024

(54) ORTHOPEDIC JOINT DEVICE

(71) Applicant: OTTO BOCK HEALTHCARE PRODUCTS GMBH, Wien (AT)

(72) Inventors: Roland AUBERGER, Wien (AT); Thomas West PICKERILL, Wien (AT); Christian BREUER-RUESCH, Wien (AT)

(21) Appl. No.: 18/577,366

(22) PCT Filed: Jul. 8, 2022
(86) PCT No.: PCT/EP2022/069085

§ 371 Date: Jan. 8, 2024

(30) Foreign Application Priority Data
Jul. 9, 2021 (DE) 10 2021 117 839.8

(51) Int. Cl. A61F 5/01; A61F 2/50; A61F 2/70

(52) U.S. Cl. CPC A61F 5/0102; A61F 2/70; A61F 2002/503; A61F 2002/5033; A61F 2002/5043; A61F 2002/5072; A61F 2002/701; A61F 2005/0155; A61F 2005/0158; A61F 2005/0167; A61F 2005/0169; A61F 2005/0179

(57) ABSTRACT

The invention relates to an orthopedic joint device having an upper part (10), a lower part (20) mounted thereon so as to be pivotable about a pivot axis (15), and a blocking device (30) which blocks a pivoting movement of the upper part relative to the lower part, wherein the blocking device never blocks in one direction of pivoting and, in the opposite direction of pivoting, can be switched from a release position to a blocking position, wherein the blocking device is assigned an actuation element (40) that holds the blocking device in the release position or moves it to the release position.

Orthopedic joint devices are in particular orthoses or prostheses which have an upper part and a lower part mounted in an articulated manner on the upper part. In the case of orthoses, the upper part and the lower part are fastened to a still existing limb, for example by shells, straps, belts, cuffs or other fastening devices. Orthoses can be used to guide movements, to limit pivoting about a joint axis, to prevent pivoting movements or to support or define an alignment of limbs with each other. In addition, orthoses can be provided with braking or damping elements in order to damp or block a pivoting movement about the joint axis. The braking or damping devices can be provided with a control so that, depending on sensor data, a changed damping in the flexion direction and/or extension direction can be provided or the movement can be. It is also known to assign force accumulators to the upper part or the lower part, so that movement support can be effected by release of the stored energy from the force accumulator.

Prostheses replace a non-existent or no longer existent limb and serve to provide a functionality that is as close as possible to the functionality of the natural limb. In addition, prostheses serve to provide the most natural appearance possible for the prosthesis user. A prosthesis upper part is formed, for example, as a prosthesis socket or as a component attached to a prosthesis socket, with the prosthesis socket serving for attachment to a limb or a limb stump. The prosthetic joint, for example a prosthetic knee joint, a prosthetic ankle joint or a prosthetic elbow joint, connects the upper part to a lower part, which in turn can have further prosthetic components, for example a lower-leg tube, a prosthetic foot or a prosthetic hand.

Particularly in the case of orthotic knee joints and prosthetic knee joints, there are arranged, between the upper part and the lower part, dampers, in particular hydraulic dampers, or brake and/or resistance devices which, on the basis of sensor data, provide different resistances in individual gait situations or completely block a movement. Such resistance devices are often designed as hydraulic linear actuators that provide a defined resistance to a flexion movement or extension movement. The resistance is changed by a change to the position of valves. When the flow cross section decreases, the corresponding resistance against movement increases. By blocking a connection line via a control valve, a movement can be blocked. Such control valves, which are used for example for stance phase damping, can be set mechatronically via servo valves or mechanically via a throttle valve. Alternatively to a hydraulic linear actuator, it is also possible to use other linear actuators, for example electromechanical linear actuators, or linearly acting locks.

Increasing the flexion resistance to the extent of blocking the joint device may be necessary for various reasons. Depending on the design of the orthopedic joint device, blocking takes place purely mechanically, for example by a blocking element latching in a recess, or by another positive or frictional blocking of the upper part relative to the lower part. Alternatively, an adjustable resistance can be increased to a level that makes a further movement practically impossible, for example by closure of a valve, by amplification of a magnetic field to change a viscosity of a magnetorheological liquid, or by a corresponding increase of an electromechanical resistance in a generator principle of a motor. However, complete blocking of the joint device in both directions of movement may also be disadvantageous, especially if this is also associated with the blocking of an extension movement.

The object of the present invention is therefore to make available an orthopedic joint device with which enhanced safety during use can be provided by simple means.

According to the invention, this object is achieved by an orthopedic joint device having the features of the main claim. Advantageous embodiments and further developments of the invention are disclosed in the subclaims, the description and the figures.

In the orthopedic joint device having an upper part, a lower part mounted thereon so as to be pivotable about a pivot axis, and a blocking device which blocks a pivoting movement of the upper part relative to the lower part, provision is made that the blocking device never blocks in one direction of pivoting and, in the opposite direction of pivoting, can be switched from a release position to a blocking position. With an orthopedic joint device of this kind, it is possible to permit blocking in one direction in any position of the upper part relative to the lower part, while the movement in the opposite direction is always possible. In one embodiment as a knee joint in an orthosis or prosthesis, the blocking of the joint advantageously takes place in the stance phase, wherein the blocking in the flexion direction during the swing phase or for initiation of the swing phase is canceled. This results in stance-phase-controlled blocking of flexion with a swing phase enabled.

The blocking device is assigned an actuation element which holds the blocking device in the release position or moves it to the release position. This actuation element ensures that the blocking device is either in the release position in the basic setting or is moved to the release position after a control signal is received or due to a certain load, a corresponding force on the actuation element or a control element or in a certain movement situation or load situation. If the actuation element holds the blocking device in the release position, the basic setting is the movable setting, such that a pivoting movement in the first direction of pivoting, for example in the flexion direction in the case of a knee joint, is released as well as in the opposite pivoting direction, i.e. the extension direction. Only when a corresponding load situation or movement situation is detected is the blocking device moved from the release position to the blocking position, so that the pivoting movement of the upper part relative to the lower part is blocked. The actuation element can also move the blocking device to the release position when the blocking device is in the blocking position, for example when the joint device has been blocked on account of a certain load situation and the load situation is no longer present. The actuation element can be, for example, a lever, a pin, a pulling element, an eccentric, a link or another force transmission device.

In one embodiment, the actuation element is assigned an actuator, which holds the actuation element in the release position or moves it to the release position, wherein the release position of the actuation element is the release position of the blocking device. In the release position of the actuation element, the upper part is movable relative to the lower part in the pivoting direction in which the blocking device can block the orthopedic joint device. The actuator can be, for example, a magnetic switch, motor, switchable spring accumulator, pneumatic element, hydraulic actuator, Bowden cable, solenoid and/or another element that supplies energy to the actuation element and moves it to or keeps it in the release position.

In addition to the actuator, a mechanical switching element can also be provided in order to manually keep the blocking element permanently in one of the states, either in the blocked position or in the released position. In the case of an electronic or electromechanical actuation, this can be effected via manually operated control elements, remote controls or settings in external appliances, for example a smartphone. The permanently released state is particularly relevant when fitting the orthosis or when cycling. The permanently blocked state is advantageous for activities in which reliable stance stability is important.

In one embodiment, the blocking device has at least one blocking element, which is mounted pivotably or slideably on the upper part and can be engaged with a blocking region on or in the lower part, or, conversely, is mounted on the lower part and can be engaged with a blocking region on or in the upper part. In this way, a mechanical coupling between the upper part and the lower part is effected, and a pivoting movement in one pivoting direction is correspondingly blocked. The blocking region and/or the bearing of the blocking elements or of the blocking element are designed in such a way that no blocking effect occurs in the opposite pivoting direction and a pivoting movement and a relative movement of upper part to lower part is always possible.

The blocking element can be formed as a blocking bracket, blocking angle, blocking wedge, brake lining, blocking hook or blocking pad, which in the blocking position bears on the blocking region and is held in contact there, either via frictional forces or positive locking, and causes blocking of the pivoting movement in one direction. For example, a blocking bracket can be mounted eccentrically with respect to a pivotable friction surface of a blocking region and can slide along it. In one embodiment, the blocking element is in frictional contact with the blocking region. In the first direction of pivoting, the blocking bracket is raised by the frictional forces and held in the release position; in the other direction of pivoting, a self-reinforcing blocking of the joint device is effected and the upper part is locked relative to the lower part. Blocking pads or blocking wedges, which bear pivotably or deformably, for example, on the upper part and slide along a friction surface on the blocking region, wedge themselves in one pivoting direction and block the joint and slide along the blocking region or the friction surface in the other pivoting direction without affecting or blocking the pivoting movement. By these blocking elements lifting away from the friction surface, movement in both directions can be released. Advantageously, the blocking elements are designed such that they lift mutually in a kind of chain reaction, provided that the actuation mechanism for enabling the movement in both directions has been activated.

In one embodiment, the blocking element is prestressed elastically in the direction toward the blocking region, and it is thus ensured that a frictional contact or a physical contact between the blocking element and the blocking region is in principle always present. This ensures that the blocking element can at any time reach a blocking position in the event of a corresponding load and movement or a corresponding switching of the actuation element or of the actuator, so that the joint is safely blocked in the respective pivoting direction without time delay. In addition, these prestressing elements can compensate for changes in shape, e.g. due to temperature, humidity or wear.

In one embodiment, the elastic prestressing of the blocking element is effected by a prestressing element which is elastically formed or elastically mounted, wherein optionally the stiffness of the elastic prestressing element and/or its prestressing is adjustable. A contact force on the blocking region or on a blocking surface can be adjusted in this way. In the case of a combination of the blocking element with an actuation element or an actuator, the prestressing element defines or influences those actuation forces which are necessary to move the blocking device or the blocking element to the release position or to keep it in the release position. This means that the orthopedic joint device can be adapted to the respective application or that wear compensation can be applied.

In one embodiment, an elastic element is mounted upstream of the blocking device, the stiffness and/or prestressing of the elastic element being adjustable. In one embodiment, the adjustment of the stiffness and/or prestressing is also carried out via an adjustable support, for example a displaceable pin or a displaceable bolt, via which the effective length of the elastic element is changed and/or the prestressing is changed. When the effective length is shortened, the stiffness of the elastic element increases; the stiffness decreases when the effective length is lengthened. The pre-stressing of the elastic element can also be adjusted by shifting one of the support points of the elastic element in the direction of action thereof. By shifting the support point in two different directions, both the stiffness and the prestressing of the elastic element can be influenced.

The blocking element can be mounted on a coupling element which is rigidly or elastically connected to the upper part or the lower part. The outer contour of the coupling element does not necessarily have to be round. In one embodiment, this coupling element is provided with an overload coupling. For this purpose, the blocking device can be equipped with an overload protection which, in the event of overloading of the orthopedic joint device, has the effect that the blocking element slips through and thus the upper part can be pivoted relative to the lower part. For this purpose, for example, starting from a certain load, the blocking element can slide along a blocking region, for which purpose the material and the surface quality of both the blocking element and the blocking region are selected accordingly. The overload protection can also be realized by a clamping element, whereby a parallel force-fit arises in the event of overload.

In one embodiment, the blocking device is guided via a central bearing or mounted on a central bearing. The central bearing or the central bearing point can be in direct contact with the upper part and with the lower part, such that, for example, a pin of a first part is mounted rotatably with a bushing or sleeve of a second part via a rolling bearing or a plain bearing. The blocking device can also be mounted on the central bearing and can be in direct contact with the upper part and lower part. In particular, the blocking device between the upper part and the lower part is arranged between two sleeve-like components, wherein the arrangement is chosen in particular such that the central bearing is arranged around a pin of a first part, about which a sleeve of the second part abuts or slides along. Bearing or engaging on the outside of the sleeve of the second part is the blocking device which, on the inside, has a friction surface or a corresponding contact surface for the blocking device on the first part.

The force required on the actuation element for switching to the free state is advantageously low, provided that no load is applied counter to the blocking direction. If, on the other hand, a load is applied counter to the blocking direction, the force required for switching to the free state increases on the actuation element; in particular, the required force increases as the load increases.

The force applied by the actuator to the actuation element may be limited or able to be limited, in particular able to be adjusted. This ensures that there is no unwanted switching by the actuator at or above a certain load level. On account of the power limitation, the actuator is not able to switch to the release position under a critical load, which ensures increased safety.

The fact that the force to be applied by an actuator at the actuation element can be adjusted and limited can be used for controlling or adapting the orthopedic joint device. The set actuator force, which can also be achieved through the selection of the actuator, ensures safety against unintentional switching to the free state under loading counter to the blocking direction. The load state of the blocking device is recorded or estimated, for example, on the basis of the state or the energy consumption of the actuator. This type of control is independent of the specific structure of the orthopedic joint device.

In one embodiment, the blocking device has an outer ring and an inner ring, between which the at least one blocking element is arranged. The blocking element or the blocking elements are mounted displaceably between the inner ring and the outer ring and can be adjusted in terms of position and/or orientation relative to the outer ring or inner ring.

In one embodiment, the outer ring is elastic or slotted, wherein the slot extends in the radial direction and breaks through the circumference of the outer ring. This makes it possible to change the inner circumference of the outer ring. In particular, if the outer ring is formed from an elastic material, it is possible to change the circumference and/or the prestressing by means of a prestressing device or an adjustment device. If the prestressing device is tensioned, the internal diameter of the outer ring can be reduced or the prestressing of the outer ring can be increased. When the outer ring is relaxed, the inner circumference is increased and the prestressing is reduced. By changing the inner circumference and/or the prestressing, it is possible, with a correspondingly elastic design of the outer ring and a certain elasticity of the upper part relative to the lower part, to achieve a rotation also in the blocking direction in which the blocking elements are located in the blocking position. This rotational elasticity is adjustable. Through a suitable choice of material, bearing and shaping of the outer ring, it is possible to achieve a radial expansion at least in parts, even with a closed cross section.

The elasticity of the outer ring can be adjusted through the choice of material of the outer ring and/or the wall thickness of the outer ring. Depending on the choice of material and/or the wall thickness, an increased or reduced elasticity of the outer ring is achieved. With a predetermined outer circumference in the case of a closed outer ring, the elasticity in one embodiment is adjusted through the choice of the internal diameter of the outer ring. Alternatively or in addition, the elasticity of the outer ring is adjusted via the axial width. The wider the outer ring, the stiffer the outer ring or the lesser the elasticity.

In one embodiment, the outer ring, in particular if slotted and provided with an open cross section, is embedded or fastened in a manner secure against rotation in a housing or another component of the upper part or lower part. This makes it readily possible to achieve an adapted change of the internal diameter or of the prestressing via an adjustment device or prestressing device. In the slotted configuration, the fastening in a manner secure against rotation is preferably effected only at one end; the opposite end of the outer ring is displaceable at least in the direction of the opposite end of the outer ring, since on the other hand no change of the internal diameter can be achieved. The prestressing device is designed, for example, as a clamping screw or clamping element, which can be operated manually or by motor.

If the inner ring moves relative to the outer ring counter to the blocking direction, the blocking elements or blocking bodies become tilted, so that they spread and become caught between the inner ring and the outer ring. The blocking elements attempt to expand the outer ring, which creates a tensile stress within the outer ring. The resulting elastic deformation of the outer ring causes the blocking mechanism to behave elastically counter to the blocking direction. Despite the blocking elements located in the blocking position, the elastic deformation of the outer ring allows a slight, elastic mobility of the upper part relative to the lower part upon further loading in the blocking direction. This avoids abrupt blocking settings, so that, for example when the block is suddenly activated, a certain elastic flexibility is achieved in order to protect the material of the orthopedic device and the patient's body. Through a suitable choice of the material and the cross section of the outer ring, i.e. the wall thickness and the width of the outer ring, the elasticity can be influenced or adjusted in order to achieve a behavior that is favorable for the respective application. Other ways of adapting the elasticity of the outer ring via the cross section are also possible. Thus, for example, the desired elasticities can be achieved through a combination of different materials or through a particular shaping by grooves, notches or the like in a uniform or non-uniform distribution over the circumference. In particular, it is advantageous for the use in artificial knee joints to adjust an elasticity counter to the blocking device, by appropriate shaping and configurations of the outer ring, to a range of between 2 Nm/° and 20 Nm/° (per angular degree of pivoting) in order to enable energy recovery, especially during the stance phase.

The elasticity of the blocking device can also be changed through the width of the geometry of the blocking elements. The elasticity of the blocking device results from the rotation of the outer ring relative to the inner ring under a given moment, which is applied to the outer ring or inner ring. Furthermore, a rotation of the outer ring arises from the rotation of the blocking elements, which is related to the expansion of the outer ring. The degree of rotation of the blocking elements depends on the gap width between the inner ring and the outer ring, which is determined by the expansion of the outer ring. Since the ratio between the rotation of the blocking elements and the gap width depends on the geometry of the blocking elements, targeted elasticities of the blocking device can be realized by adjusting the contours of the blocking elements.

In the orthopedic joint device with an upper part and a lower part, which is arranged on the upper part pivotably about a pivot axis via at least one joint, with a first actuator, which is arranged between the upper part and the lower part and has a first adjustment element, via which the movement resistance of the first actuator against a pivoting movement of the lower part relative to the upper part about the pivot axis is adjustable, and with at least a second actuator, which is arranged between the upper part and the lower part and has at least a second adjustment element, via which the movement resistance of the second actuator against a pivoting movement of the lower part relative to the upper part about the pivot axis is adjustable, with at least one sensor and with a control unit which is connected to the at least one sensor for transmitting sensor data and controls the first adjustment element and the second adjustment element in accordance with sensor data, provision is made in one embodiment that the control unit controls the first adjustment element and the second adjustment element synchronously or is configured to control the adjustment elements synchronously. In the case of a synchronized control, the control unit adapts the control of the adjustment elements to one another in time or coordinates them with one another such that, in particular, synchronized release and blocking of the pivoting movement of the lower part relative to the upper part is permitted. A synchronized control of this kind prevents overloads caused by pivoting that is released too late or that is blocked too early. In addition, there is the advantage that an orthopedic technician does not have to carry out complex adaptations and adjustments of the resistance devices in the context of an adjustment process. Furthermore, by using a single central control unit for controlling a plurality of adjustment elements, redundancies in the sensor system and hardware are avoided, resulting in lower manufacturing costs, lower weight and an increased operational life of the orthopedic joint device. At the same time, this enables a more compact design of the orthopedic joint device. In principle, more than two adjustment elements can be controlled in a synchronized manner by one control unit.

Advantageously, the actuators are motor drives, spring accumulators, releasing and/or locking devices and/or hydraulic and/or pneumatic resistance devices, in particular rotary hydraulics. Each actuator can be arranged on a joint or itself form the latter. The adjustment element can be used to adjust the resistance of the respective actuator against a pivoting movement of the lower part relative to the upper part. In the case of a hydraulic resistance device, the adjustment element can be, for example, a control valve via which the resistance of the resistance device is adjustable. The valve can in particular be switched electrically or electronically. An adjustment element can cause a change in the resistance of the actuator both mechanically and by motor or electronically. If the actuator is designed as an electromotive drive, for example, the brushes attached to the commutator or the coils of the rotor are to be regarded as an adjustment element according to the invention.

The at least one sensor can be designed as a force sensor, angle sensor, displacement sensor, moment sensor, acceleration sensor, speed sensor or position sensor.

This sensor is configured to acquire at least one measured value and transmit it to the control unit. Depending on the sensor data, the orthopedic joint device can be controlled in order to be able to make adjustments that are adapted to the user or to the respective movement or movement situation.

A further development provides that the first actuator has a first joint, which has a first joint axis coaxial to the pivot axis, and that the second actuator has a second joint, which has a second joint axis, likewise coaxial to the pivot axis. The fact that the actuators form separate joints improves the stability and load-bearing capacity of the orthopedic joint device. The coaxiality of the joint axes with the pivot axis has the effect that no unwanted moments and/or loads arise during a pivoting movement of the lower part relative to the upper part. The pivot axis is preferably coaxial to the natural joint axis or to a compromise joint axis, in order to achieve the most natural movement behavior possible.

Preferably, the first actuator is arranged laterally and the second actuator is arranged medially. The bilateral arrangement of the actuators leads to a further increase in the mechanical load-bearing capacity of the orthopedic joint device. Particularly in the case of the bilateral arrangement of the actuators, for example in a knee orthosis, a synchronized control of the adjustment elements is required in order to avoid unwanted knee flexion and knee extension. The bilateral arrangement of the actuators additionally results in a reduction of torsional loads, which can arise in a monolateral arrangement, for example on account of the one-sided damping of the pivoting movement of the lower part relative to the upper part.

Preferably, the first actuator and the second actuator are based on different mechanical operating principles. Particularly preferably, the first actuator is designed as a motor drive or a hydraulic and/or pneumatic resistance device, in particular a rotary hydraulics device, and the second actuator is designed as a releasing and locking device. For example, it is advantageous if a motor drive or a hydraulic and/or pneumatic resistance device, in particular a rotary hydraulics device, is arranged laterally and a releasing and locking device is arranged medially. Releasing and locking devices, in particular mechanical releasing and locking devices, are simple and space-saving and allow permanent locking to be made available without energy being consumed.

In addition, this prevents the joint from flexing during prolonged periods of stance phases on account of leaks, which may be the case with hydraulic or pneumatic resistance devices. In this case, the secure stance of the user can be ensured by locking a medially arranged releasing and locking device. With an electromechanical actuator, energy can be saved in this situation, because the parallel interlocking device does not require active securing of the stance.

In a further development, provision is made that a plurality of actuators are arranged medially and/or laterally. It is possible in this way to adapt the number of actuators to the body weight of the user of the orthopedic joint device. Preferably, a modular system is used, so that quick and cost-effective adaptation of the orthopedic joint device to the body weight of the user can take place. It may also be advantageous to arrange actuators exclusively laterally, in order to achieve more freedom of movement in the medial region.

Preferably, the orthopedic joint device forms an artificial knee joint, elbow joint and/or ankle joint. The orthopedic joint device can also form a plurality of joints, in particular an artificial knee joint and an artificial ankle joint. It is also possible to form a plurality of joints of the same type if, for example, more than two joints of the same type are to be replaced or supported on two extremities.

Preferably, all the adjustment elements are controlled by a single control unit. This avoids redundancy and allows the control unit to adapt the control of all the adjustment elements to one another, allowing for a natural movement behavior and more precise, load-optimized control of the adjustment elements. Alternatively, the adjustment elements can be controlled with a plurality of control units. In an orthopedic joint device that forms a plurality of joints, it may be advantageous, for example, if the adjustment elements of each joint are controlled by a control unit assigned to the joint.

Advantageously, the adjustment elements are controlled via control lines or a bus system. Especially when a large number of devices are controlled by the control unit, the number of data lines required is significantly reduced by the use of a bus system.

In a further development, provision is made that the control unit controls a plurality of adjustment elements with the same control signal. In particular, if the actuators assigned to the respective adjustment elements are based on the same mechanical operating principle, this affords the advantage that the actuators are subjected to the same load and a homogeneous reaction behavior of the actuators can be achieved. Synchronized behavior can also be achieved with different actuator technologies by local processing of a uniform control signal at the actuator level.

Exemplary embodiments are explained in more detail below with reference to the figures. The same reference signs denote the same components. In the drawings:

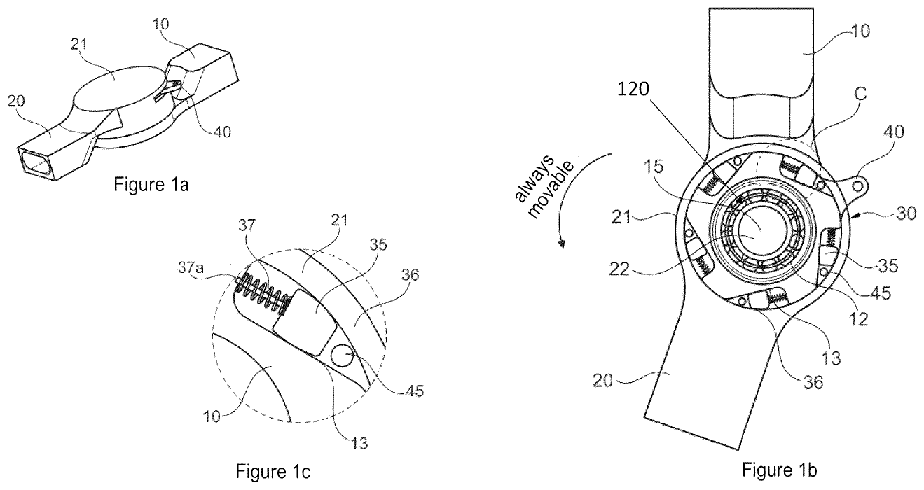

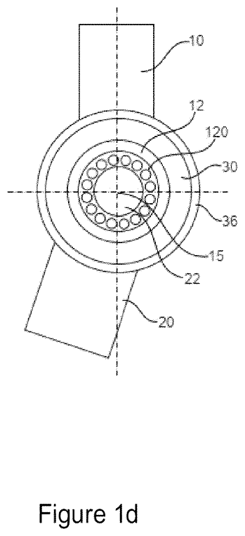

FIGS. 1a to 1c show a first embodiment of the joint device with pivotable blocking wedges;

FIG. 1d shows an abstract representation of the bearing concept;

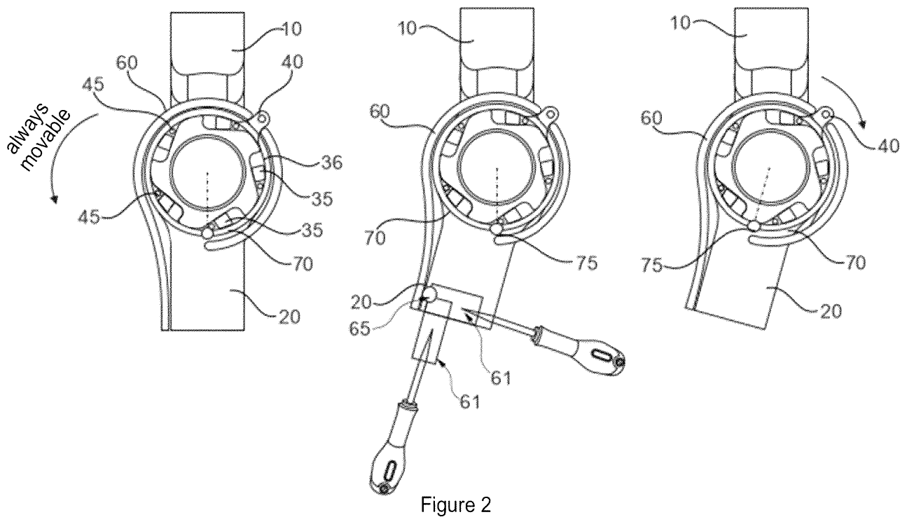

FIG. 2 shows a variant with an upstream adjustable elastic element;

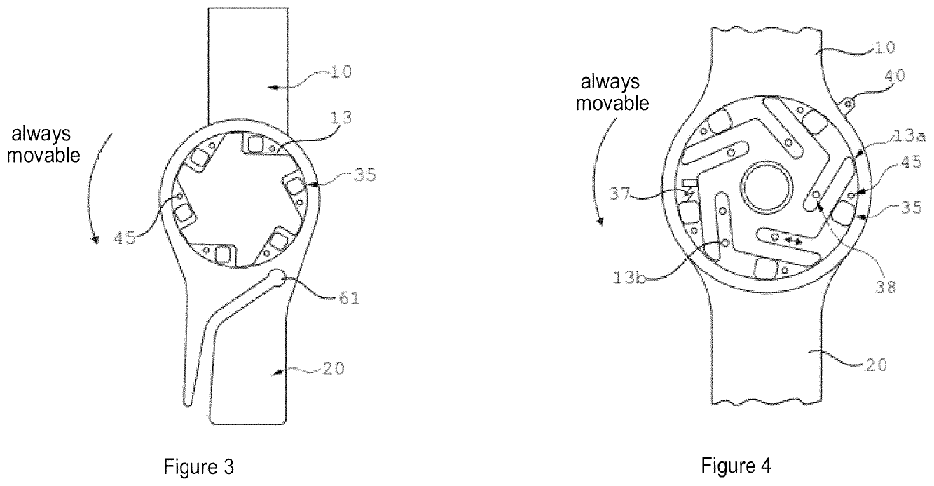

FIG. 3 shows a variant with an elastic lower part;

FIG. 4 shows a variant with integrated elasticity;

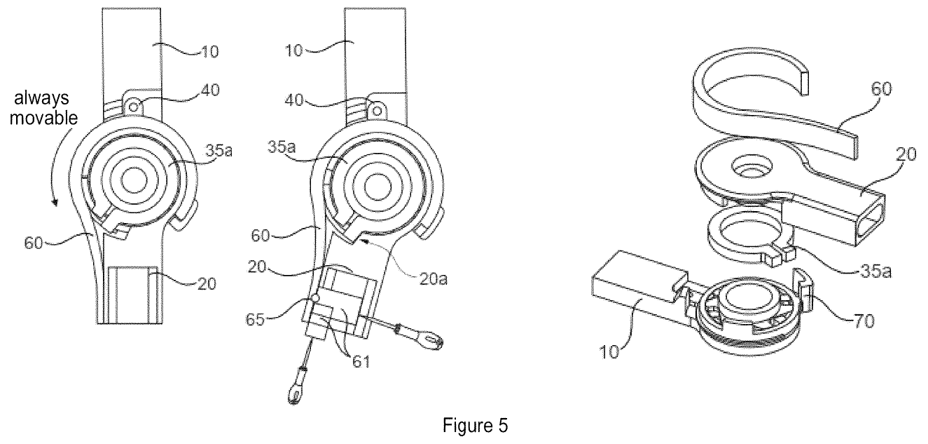

FIG. 5 shows a variant with a clamping ring;

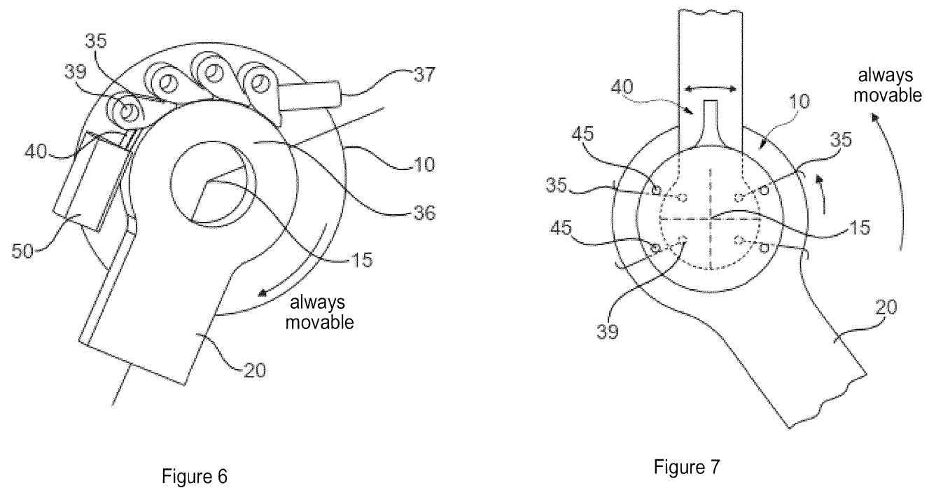

FIG. 6 shows a variant with blocking wedges;

FIG. 7 shows a variant with blocking brackets;

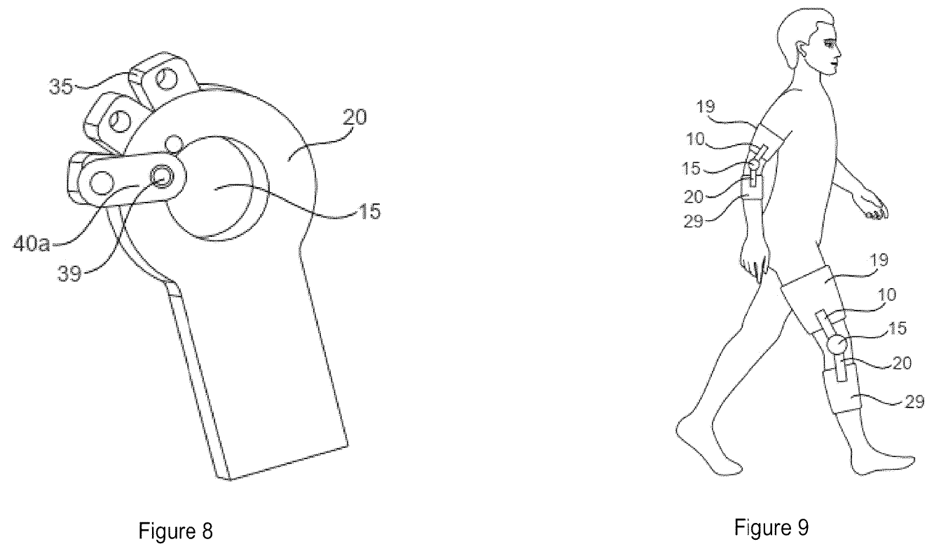

FIG. 8 shows a variant with brake linings;

FIG. 9 shows applications of the joint device;

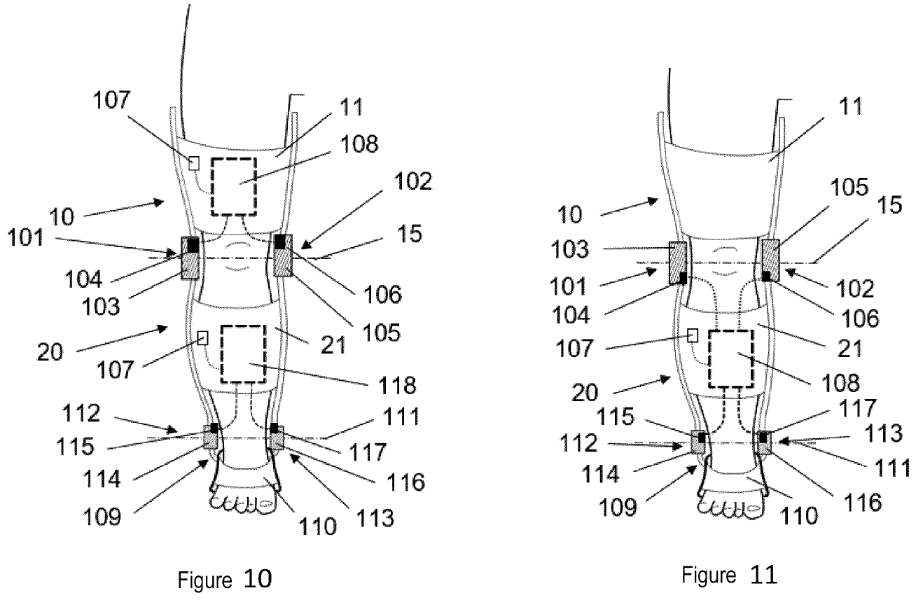

FIGS. 10 to 13 show variants of the orthopedic joint device in the state when applied to a lower right extremity, in a front view;

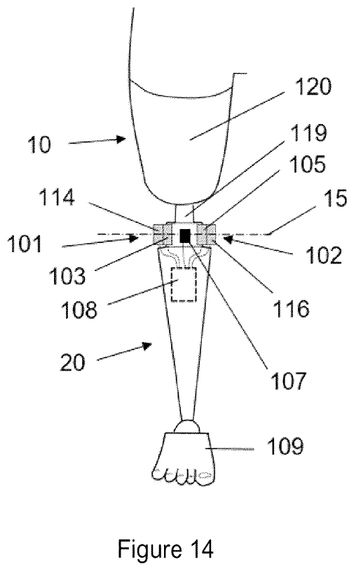

FIG. 14 shows a variant of the orthopedic joint device in a prosthetic knee joint;

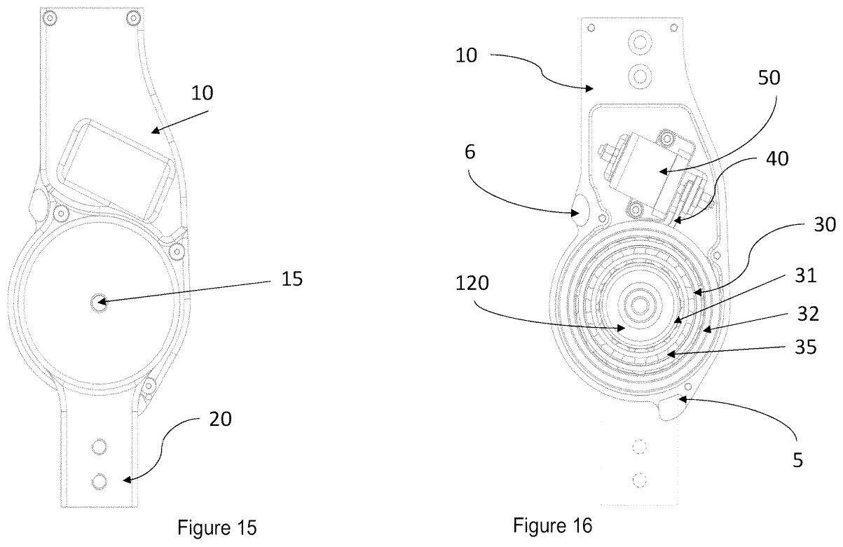

FIG. 15 shows a variant of the joint device in the form of a knee joint;

FIG. 16 shows a representation of FIG. 15 without cover;

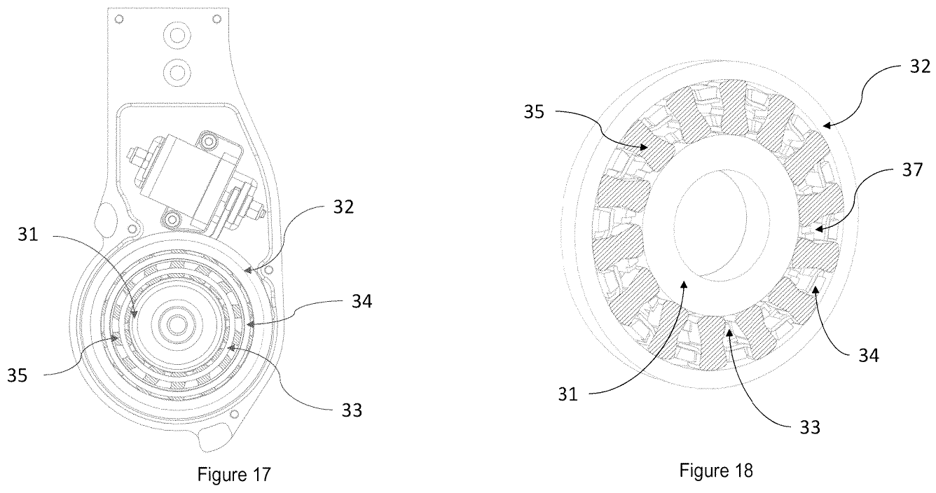

FIG. 17 shows a detailed view of the upper part according to FIG. 16;

FIG. 18 shows a sectional view through the blocking device;

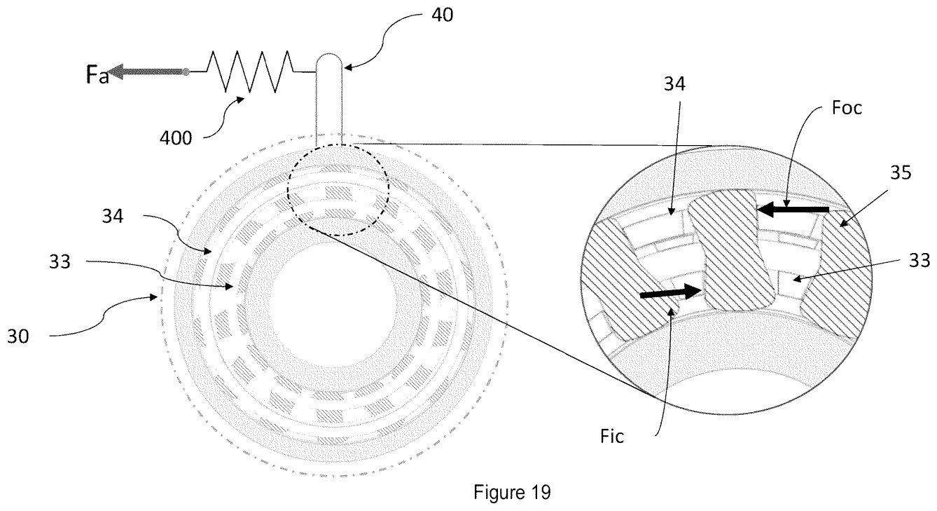

FIG. 19 shows a schematic representation of the blocking device along with a detailed representation;

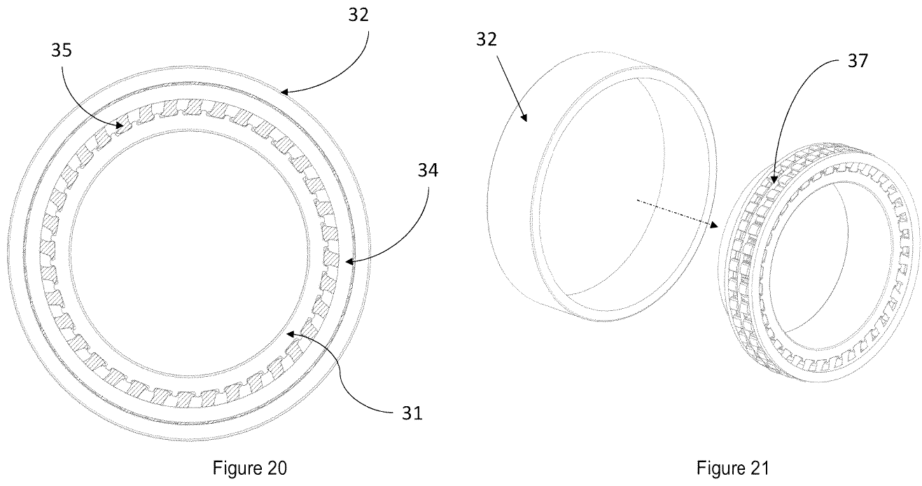

FIG. 20 shows a representation with blocking element fixation on the inner ring;

FIG. 21 shows an exploded view of FIG. 20;

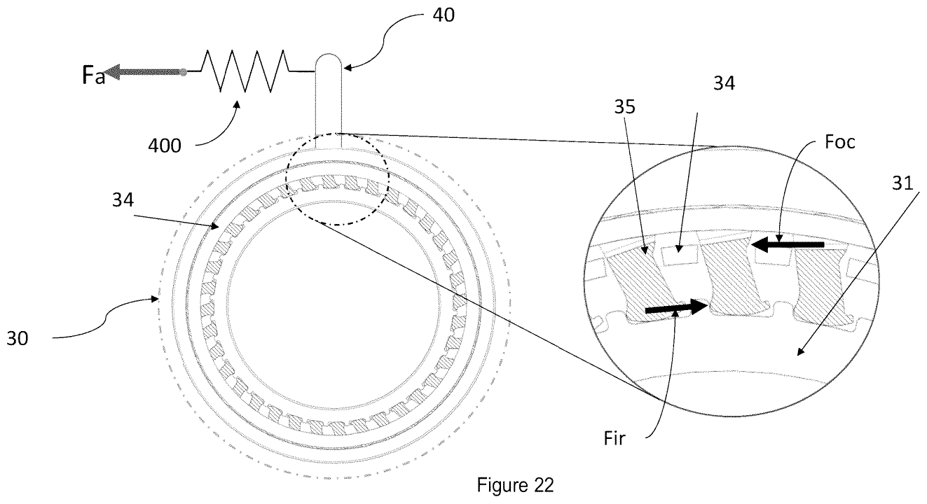

FIG. 22 shows a schematic representation of the actuation of the embodiment according to FIG. 20;

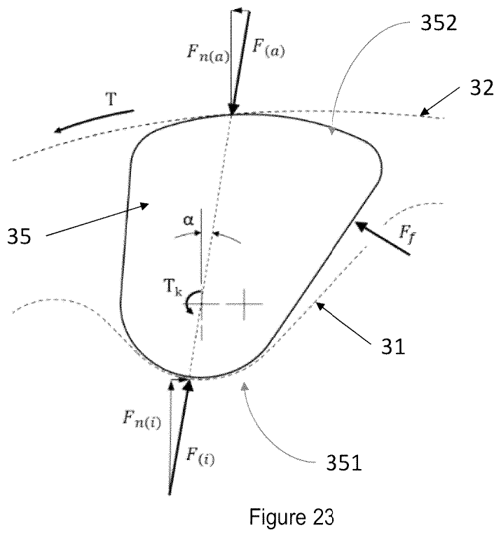

FIG. 23 shows a detailed representation of the mounting of a blocking element;

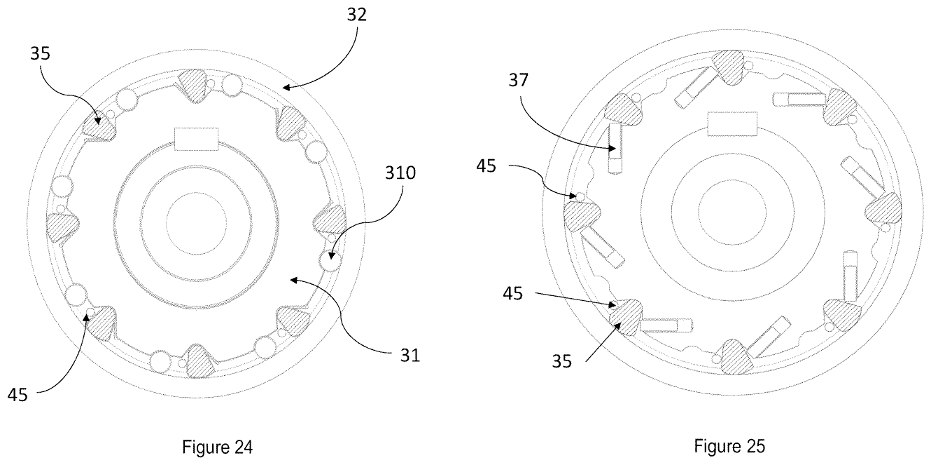

FIG. 24 shows an embodiment with blocking elements mounted on the inner ring;

FIG. 25 shows a sectional representation according to FIG. 24 with prestressing elements;

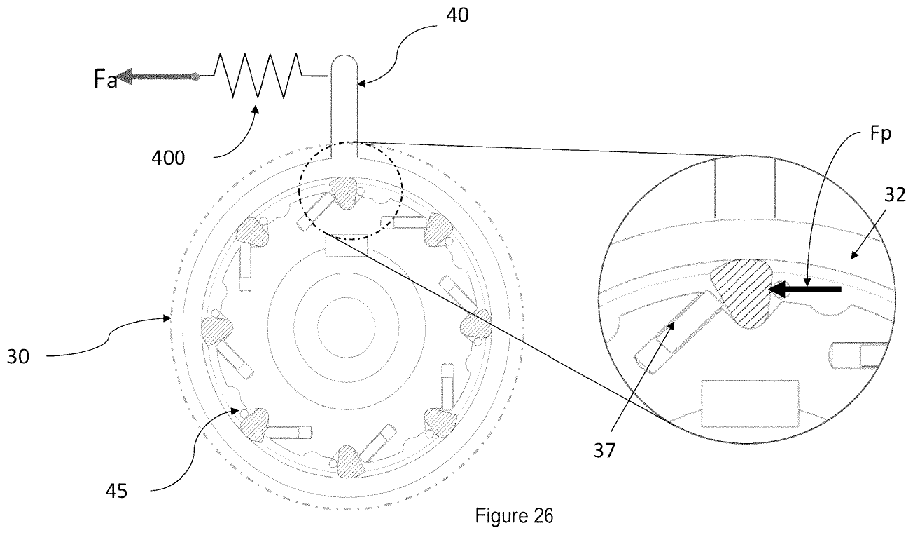

FIG. 26 shows a schematic representation of the actuation of the embodiment according to FIG. 25;

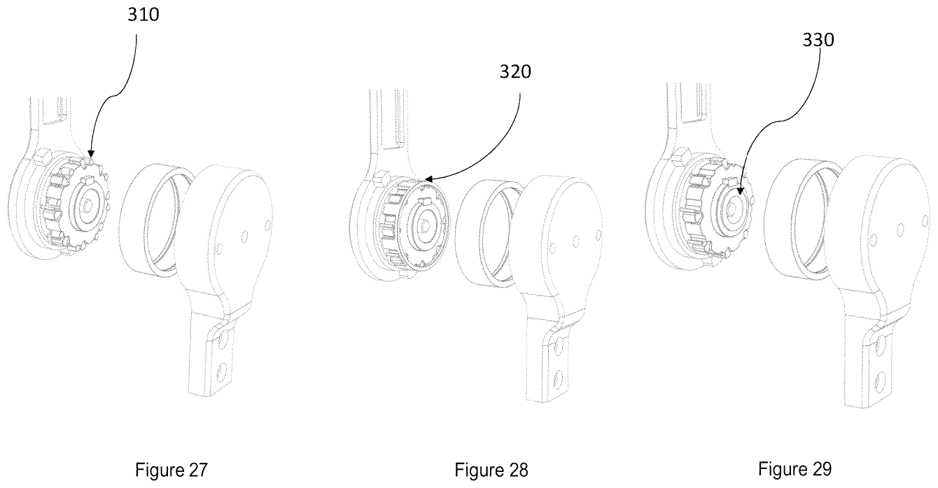

FIGS. 27 to 29 show bearing concepts;

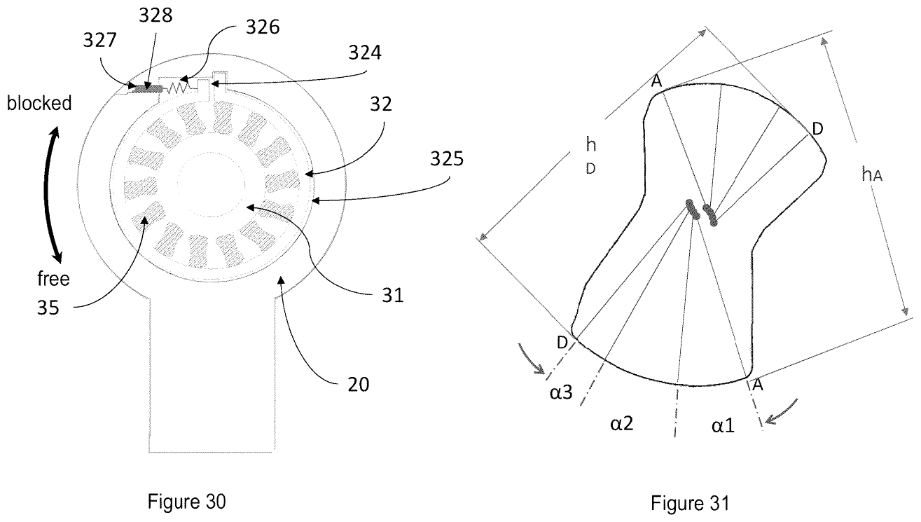

FIG. 30 shows a variant with a slotted outer ring; and

FIG. 31 shows a detailed representation of a blocking element.

FIG. 1a is a perspective overall view of an orthopedic joint device with an upper part 10 and a lower part 20, which are mounted pivotably on each other. In the illustrated embodiment, the orthopedic joint device is shown as an orthotic joint, in particular as an orthotic knee joint. The upper part 10 and the lower part 20 each have receptacles for orthosis rails which extend along a limb and are fastened to the limb with appropriate fastening devices such as shells, straps or buckles. The orthopedic joint device can also be designed as an ankle joint, an elbow joint or another joint. In principle, it is also possible and provided to equip a prosthesis, instead of an orthosis, with such a joint device. The statements concerning orthoses also apply to prostheses.

In the embodiment shown according to FIG. 1, the orthopedic joint device provides, on the upper part 10, a bearing receptacle in which the lower part 20 is pivotably mounted. The lower part has a housing 21 in which, in addition to a bearing, a blocking device is also arranged, which will be explained in more detail later. The blocking device is assigned an actuation element 40 which protrudes from the housing 21 and can be moved either by hand or by motor. The function of the blocking device is explained below.

In FIG. 1b, the orthopedic joint device according to FIG. 1a is shown in a partially transparent plan view. The lower part 20 with the housing 21 has a cylindrical inner wall. Likewise formed in the housing 21 is a pin 22, which extends perpendicular to the plane of the drawing and serves as a receptacle for a central bearing 120, a rolling bearing in the embodiment shown. This rolling bearing 120 as central bearing is arranged in a bearing receptacle 12 of the upper part 10. As an alternative to the bearing in the form of a rolling bearing, the bearing can also be realized as a plain bearing or by another bearing technology. Around the bearing receptacle 12, run-on bevels 13 are formed which extend in the direction toward the cylindrical inner wall of the housing 21. Between the run-on bevels 13 and the inner wall of the housing 21, wedge-shaped clearances are formed in which respective blocking elements 35 are arranged as part of the blocking device 30. The blocking elements 35, in the illustrated embodiment five blocking elements 35, are formed as blocking wedges and are in frictional contact with the inside of the housing 21. In order to compensate for manufacturing tolerances, the blocking wedges can be designed in a cambered shape, as shown in FIG. 1c. In order to minimize the local stresses in the material, it is advantageous to have blocking wedges that bear evenly on the run-on bevels 13 or the blocking region 36. On the inside of the housing 21, a blocking region 36 is thereby formed when the blocking elements 35 engage with the respective surface portion of the inner wall of the housing 21. If the upper part 10 is held in the position shown and the lower part 20 is pivoted counterclockwise, the lower part 20 rotates relative to the run-on bevels 13, which are formed or arranged on the upper part 10. Through the frictional contact of the blocking wedges 35 with the inner wall of the housing 21, the blocking wedges 35 are carried along, so that they move into an enlarging clearance. As a result, the frictional contact between the blocking wedges 35 and the inner wall of the housing 21 is lost or reduced sufficiently such that a counter-clockwise movement of the lower part 20 relative to the upper part 10 is possible. If an oppositely directed movement of the lower part 20 takes place clockwise around the pivot axis 15, the blocking wedges 35 wedge in the tapering clearance, such that the relative movement between upper part 10 and lower part 20 is blocked. A counter-clockwise movement of the lower part 20 is always possible; a clockwise movement of the lower part 20 is always blocked. Depending on the orientation of the run-on bevels 13, the direction of rotation in which blocking always takes place and in which movement is always possible can be set. In a mirror image design, the block would operate in the opposite direction.

In one variant, the central bearing, realized in the embodiment of FIG. 1 by a rolling bearing, can also be dispensed with, provided that there is sufficient support and guidance by the outer edges of the run-on bevels in the upper part 10 in relation to the cylindrical inner surface of the housing 21 on the lower part 20. This places high demands on the manufacturing accuracy of the components.

In order to allow the orthopedic joint device to permit a pivoting movement of the upper part 10 relative to the lower part 20 in both pivoting directions, the blocking elements 35 are each assigned an unlocking pin 45 via the actuation element 40. The actuation element 40 is formed as a disk which is mounted within the housing 21 and which can also be moved about the pivot axis 15. For this purpose, a slot is formed in the housing 21, as is shown in FIG. 1a. If the actuation element 40 is moved counter-clockwise relative to the upper part 10, the unlocking pins 45 rotate relative to the upper part 10 about the pivot axis 15 and move the blocking elements 35 toward the rear wall of the clearances which open and enlarge in this direction. They thus disengage the blocking elements 35 from the blocking region 36 or the contact surfaces of the blocking elements 35 on the inside of the housing 21, such that the joint device is released in both directions of movement.

FIG. 1c shows a detailed view C of FIG. 1b. The blocking element 35 in the form of a blocking wedge is arranged between the run-on bevel 13 of the upper part 10 and the blocking region 36 on the inside of the housing 21 of the lower part 20 and bears on both surfaces. In order to ensure contact, the blocking element 35 is assigned a prestressing element 37, which is formed as a spring in the illustrated embodiment. Tolerances and wear of the opponents can be compensated via the prestressing element 37, which is arranged around a guide 37a for the blocking element 35. In the illustrated embodiment, the unlocking pin 45 is not in contact with the blocking element 35, so that a displacement of the lower part 20 relative to the upper part 10 is possible only when the lower part 20 is rotated counterclockwise with the upper part 10 fixed. The outside of the blocking element 35 then bears on the blocking region 36. The frictional forces present cause a displacement of the blocking element 35 counter to the preload of the prestressing element 37 in the direction toward the rear wall of the clearance that opens in this direction. This cancels out a blocking effect, and the lower part 20 can be pivoted clockwise relative to the upper part 10. An opposite movement of the lower part 20 has the effect that the blocking element 35 is pressed against the blocking region 36 by the prestressing element 37 and wedges between the run-on bevels 13 and the blocking surface 36. Further movement of the lower part 20 in a clockwise direction is prevented. If greater forces are applied, the blocking effect is increased. In order to release this block, the locking pins 45 are moved counterclockwise in the direction of the blocking element 35, as a result of which the blocking element 35 disengages from the blocking region 36 and, in this way, free mobility is possible both clockwise and counterclockwise. The blocking element is guided by an optional, additional guide element 37a in order to prevent unintentional tilting in the released state.

The actuation element 40 can be held permanently in a release position, so that a free mobility of the upper part 10 relative to the lower part 20 is always possible.

Such free mobility may be desired, for example, when a person is in a seated position. The actuation element 40 can be fixed in the release position. If the fixing is canceled, for example if the person wants to stand up, an extension movement is always possible. A flexion movement is automatically blocked and is only canceled if the blocking device 30 with the actuation element 40 and the release pins 45 are rotated accordingly. A manual switch can be used to implement both permanent enablement and permanent activation of the flexion block. Alternatively or in addition, the enablement can also be effected automatically, for example according to the load, in particular when a predetermined extension moment is exceeded, or electronically via an actuation actuator depending on sensor signals or user commands, e.g. via control elements, a remote control or smartphone app.

FIG. 1d shows an abstract representation of FIG. 1 in order to explain in detail the concept of the concentric arrangement of central bearing 120 and blocking device 30. This arrangement ensures that the blocking elements (not shown) of the blocking device 30 are free of lateral loads and tilting, since the functions “bearing” and “switchable blocking device” are taken over by various components. Thus, a safe function and reliable activation can be ensured by the actuation mechanism 40 (not shown). By means of the central bearing 120 shown schematically as a rolling bearing, the bearing receptacle 12 assigned to the upper part 10 and the pin 22 assigned to the lower part 20 are mounted rotatably to each other. The mobility of the upper part 10 relative to the lower part 20 is additionally influenced by the blocking device 30, which is arranged between the bearing receptacles 12 of the upper part and the blocking region 36 on the lower part 20. Depending on the state of the actuation element 40 (not shown), the blocking device 30 permits a movement in either one or both directions, wherein the configuration of the blocking element is arbitrary. Instead of a rolling bearing, other bearing technologies, in particular plain bearings, are also conceivable for the central bearing 120.

FIG. 2 shows an embodiment in which the coupling element 70 is designed as a ring element. A spring 60, which is designed as a leaf spring in the illustrated embodiment, is fixed to the ring element 70 at a fastening point 75. In the left-hand illustration in FIG. 2, the upper part 10 is locked with the lower part 20 via the blocking device 30 with the blocking elements 35; free mobility of the ring element 70 relative to the upper part 10 in the clockwise direction is not provided. The ring element 70 is supported on the lower part 20 by the spring 60. On account of the elastic properties of the spring 60, a slight pivoting of the lower part 20 relative to the upper part 10, as shown in the central illustration in FIG. 2, is also possible with the blocking activated. As soon as the blocking elements 35 are disengaged via the actuation element 40, the blocking effect is canceled and the lower part 20 moves together with the ring element 70 relative to the upper part 10. This can be seen in the right-hand illustration in FIG. 2 at the pivoted fastening point 75. Since the fastening point 75 is freely movable in both directions when the blocking device is enabled, the spring 60 has no effect in this situation, as a result of which an unimpeded movement of the lower part 20 relative to the upper part 10 is provided.

In the central illustration in FIG. 2, two adjustment devices 61 are shown for the spring prestressing and the spring stiffness of the spring 60. Using these adjustment devices 61, it is possible to position the support point of the support 65 of the spring element 60 on the lower part 20 differently. If the support point 65 is shifted further to the left in the illustrated embodiment, the spring prestressing increases; if the support point 65 is moved further in the direction of the pivot axis 15, the stiffness increases on account of the shortened effective spring length. Alternatively, the support point can also be displaced by inserts, in particular sliding blocks or wedge-shaped spacers.

FIG. 3 shows another variant, in which the locking is carried out according to the principle of FIGS. 1 and 2. In this example, the blocking acts in a clockwise direction when the lower part 20 is rotated relative to the upper part 10. In the embodiment, instead of the five blocking elements 35 shown above, six blocking elements 35 are arranged uniformly about the circumference of the cylindrical inner surface of the lower part 20. Accordingly, six release pins 45 are provided, which can be rotated via an actuation device (not shown) in order to disengage the blocking elements 35. The number of blocking elements 35 and release pins 45 may differ from this and vary as desired. In order to permit, as in FIG. 2, a pivoting movement of lower part 20 relative to upper part 10 that is limited by spring force, even when the joint is locked and the pivoting movement about the pivot axis 15 is blocked, the lower part 20 is configured elastically in the illustrated embodiment. This is done by introduction of a slot 61 within the lower part 20 and by the elastic configuration of the material or by arrangement of an elastic element in the slot 61. A flexibility of upper part 10 to lower part 20 is thus provided by a corresponding flexible design of the upper part 10 or lower part 20. The slot 61 forms a joint or hinge in the lower part 20, wherein the maximum pivotability of upper part 10 or lower part 20 within the joint or hinge is defined by the slot 61 via the width of the slot. The elastic properties of the lower part 20 can be varied by different materials or geometries of elastic bodies that can be inserted into the slot 61. Alternatively or in addition, the upper part 10, as already mentioned above, can also be provided with a slot or configured elastically. A greater elastic deflection can be achieved by an elastic design of both the upper part 10 and the lower part 20. Furthermore, in this case, the deformations are divided between both components, which results in lower loading of the materials.

If the release pins 45 are in engagement with the locking elements 35, a free pivotability of upper part 10 to lower part 20 is possible, regardless of a flexible and elastic design of the lower part 20 and/or upper part 10. Such an embodiment permits a serial arrangement of elasticity and flexibility together with locking in one direction of pivoting and a permanent mobility in the opposite direction of pivoting.

Another variant is shown in FIG. 4, in which the run-on bevels 13 are in the form of resilient arms 13a. In the illustrated embodiment, five arms 13a are formed, which extend to the inner wall of the upper part 10, although they may also be shorter. In the illustrated embodiment, a cylindrical friction surface, hence the blocking region 36 for the blocking elements 35, is provided in the upper part 10. Arranged between the run-on bevels 13 of the arms 13a are the blocking elements 35, to which release pins 45 are assigned, which are again mounted rotatably about the pivot axis 15 on the actuation element 40, in order to permit free pivotability in both directions of pivoting. If the release pins 45 are not in contact with the blocking elements 35, a free movement of the lower part 20 with respect to the upper part 10 is then only possible counterclockwise. In the opposite direction, the blocking elements 35 cause bending of the resilient arms 13a, as a result of which an elastic resistance is realized. The elastic deformation of the resilient arms 13a can be limited by a defined support point 13b, whereby the elastic force and thus the elastically applied moment about the axis of rotation are limited, and at higher moments, further movement is blocked. The support point 13b can be integrated into the component, which also contains the spring elements, as a limitation by means of appropriate shaping.

In the embodiment shown, the inside of the resilient arms 13a is assigned pins as supports 38, which are mounted pivotably or adjustably along the arms 37, as is indicated by the double arrow. The pins serving as supports may also not be provided. If the pins 38 are moved further toward the end of the slot below the arms 13a, the stiffness of the arms 13a decreases. If the pins 38 are moved further to the outer end of the arms 13a, the stiffness of the arms 13a increases The arms 13a thus work as bending beams, the elasticity of which is adjustable. In addition to the elasticity of the arms 13a, the elastic elements 37 may be provided for acting on the blocking elements 35 in the direction of a closed position. The elastic elements 37, which act in the circumferential direction or along the longitudinal extent of the arms 37, permit a uniform contact pressure of the blocking elements 35 on the blocking region 36 or the inner wall of the upper part 10 and also tolerance compensation and wear compensation. The elastic design of the arms 13a supports this effect; in addition, in the case of a blocked joint, the braking is is cushioned when the arms 13a deform slightly due to their elasticity.

FIG. 5 shows a variant with a clamping ring 35a which also acts as overload protection. Once again, a movement of the coupling element 70 for spring mounting relative to the upper part 10 is possible only in the counterclockwise direction. It is blocked in the clockwise direction, unless the coupling element or ring element 70 is released for movement via the actuation mechanism 40. Under normal load, the clamping ring 35a is only in loose contact with the upper part 10, so that a relative movement is possible. At high load, the clamping ring 35a is tightened by the contact of the lower part 20 with the clamping ring 35a at the point 20a, as shown in the middle figure, thereby creating an additional force transmission point between the upper part 10 and the lower part 20. By means of the load-dependent additional force-fit, the blocking elements 35 can be designed for lower loads, and thus made more compact, because occasionally occurring overloads are absorbed. In addition, FIG. 6 shows devices 61 for adjusting the spring properties. By virtue of these devices, the support point 65 of the spring 60 on the lower part 20 can be moved, as a result of which the prestressing and stiffness of the spring 60 can be adapted. The spring prestressing and stiffness can be adjusted, as described above, via the adjustment devices 61. This can be done, for example, by means of a screw, an eccentric, insert parts or another device or method. Motor adjustment is also conceivable.

FIG. 6 shows another variant, in which the upper part 10 is depicted only schematically. On a circular outer surface of the lower part 20, brake wedges mounted eccentrically to the pivot axis 15 are mounted as blocking elements 35 on axes 39 with the upper part 10. The individual brake wedges are arranged on top of one another in an overlapping manner. In order to compensate for tolerances and wear, the individual brake wedges can also be connected via elastic elements. On the brake wedge on the far right, a spring element or elastic element 37 is arranged which presses the brake wedges in the direction of the outer circumference of the circular end-piece of the lower part 20. The outer circumference of the end-piece of the lower part 20 forms the blocking region 36 or the blocking surface, which engages with the brake wedges. The contact surfaces of the brake wedges are located next to a connecting line of the respective axis 39 to the pivot axis 15, such that a pivoting movement of the lower part 20 is possible only in one direction of pivoting, in the illustrated embodiment only in the clockwise direction. If the lower part 20 is moved counterclockwise about the pivot axis 15, the blocking elements 35 wedge on the contact surface or the blocking region 36 and prevent further rotation. In order to cancel this blocking effect, an actuation element 40 in the form of a slide is provided, which is positioned below the axis 39 of the left-hand blocking element 35 and lifts the latter from the blocking surface or the blocking region 36. On account of the overlapping or elastically connected arrangement of the four blocking elements 35, all of the blocking elements 35 are lifted and a pivoting movement is enabled. The actuation element 40 is actuated via an actuator 50, for example a lifting magnet, magnetic actuator, a piezo stack, a motor or another drive. The actuation can be controlled via a sensor or a plurality of sensors or can be activated manually and individually via a switch. As an alternative to the overlapping design of the brake wedges, by which both the force of the actuator 50 and the force of the prestressing element 37 are divided between the brake wedges, the latter can also be designed separately from one another, in which case a structure for transmitting the force of the actuator 50 or a prestressing element 37 is provided individually for each wedge.

A further variant is shown in FIG. 7 in which, instead of wedge-shaped blocking elements 35, brackets are mounted pivotably on the upper part 10. The brackets 35 engage around the outside of the lower part 20, wherein the bearing 39 is eccentric to the pivot axis 15. The lower part 20 is freely movable when pivoting counterclockwise. In the case of clockwise pivoting, the brackets as blocking elements 35 block a further movement since, on account of the eccentric bearing and the guiding on the outer surface of the lower part 20 with friction during clockwise pivoting, they are pressed against the outside of the lower part 20 and cause locking. The actuation element 40 with the release pins 45 works as described above; when the actuation element 40 rotates counterclockwise, the brackets 35 are lifted from the lower part 20. This results in an unblocking, so that free mobility of upper part 10 to lower part 20 is ensured in both directions. In the case of a blocked joint, an elastic design of the brackets 35 can be used to achieve an elastic behavior counter to the blocking direction.

FIG. 8 shows a further variant, in which the bracket is replaced by force transmission structures 40a which are connected to the actuation element 40 and are connected to the blocking elements 35, e.g. brake linings. These structures are mounted eccentrically with respect to the axis of rotation 15 on the upper part 10 (not shown) via bearing points 39. If the lower part 20 pivots in the clockwise direction, the blocking elements 35 lift away from the lower part 20 on account of the frictional forces via the force transmission structure 40a, and free pivotability is provided. In the reverse direction of pivoting, the blocking elements 35 are pressed onto the outer circumference of the lower part 20 and blocking is effected. By means of an actuation element (not shown) connected to the force transmission structure 40a, the blocking elements 35 can be permanently lifted from the lower part 20, as a result of which a free movement is possible in both directions of pivoting. Advantageously, the blocking elements 35 or force transmission structures 40a are here also subjected to a slight prestressing force in the direction of the lower part 20, in order to ensure secure blocking and wear compensation. An elastic design of the force transmission structures 40a can achieve an elastic behavior in the blocking direction.

FIG. 9 shows two examples of the use of the orthopedic joint device in the form of knee orthoses and elbow orthoses. Both orthoses have a upper part 10 and a lower part 20, which are mounted on each other in an articulated manner about the pivot axis 15. The upper part 10 is fastened to the upper arm with the first fastening member 19, for example a cuff, a shell or straps, while the lower part 20 is fastened to the forearm via a second fastening member 29. The second fastening element 29 is suitably formed, for example likewise as a shell, partial shell, cuff or the like. Between the upper part 10 and the lower part 20, the joint device is arranged with a blocking device as described with reference to the preceding figures.

With all orthopedic joint devices, as have been shown above, mechanical protection against bending in one direction is provided, as long as no measures are taken to cancel the blocking effect in the predetermined, critical direction of pivoting. The blocking remains in place even under load and is reinforced by positive feedback in the event of an increasing load. This means that the blocking is also retained if electronic components fail on account of a fault or lack of power supply.

Alternatively, the blocking elements can be subjected to a prestressing force in the other direction, such that they have to be brought actively into engagement. In this configuration, the joint is free in the non-energized, non-actuated state. This may be undesirable for use in prosthetic or orthotic knee joints. However, when used in elbow joints, such behavior is advantageous, since it can ensure an unimpeded swing of the arm, e.g. when walking, and the blocking is activated only if it is actually needed.

An actuator or an actuating device serves to cancel the friction and thus also to cancel the blocking when the latter is no longer required. Advantageously, blocking leads to activation of a serially arranged spring device, whereby a shock-like loading of the blocking can be cushioned. In addition, an elastic intermediate storage of the user's kinetic energy can be realized, e.g. during stance phase flexion when walking. It is possible to block the joint device in one direction of movement or direction of pivoting in any position; free pivotability in the opposite direction is always possible.

A feature of all of these designs is that they can be switched to the free state with a low actuator force, provided there is no loading counter to the blocking direction. With loading in the blocking direction, the switching force required for this increases sharply. This can be used as safety against unintentional switching under loading, for example by limiting the force of the actuator for actuation of the switching. This can simplify the control of an orthotic or prosthetic joint based on the joint device. On account of this property, it is possible in many cases to dispense with additional sensors for measuring the load state, since this can also be estimated, for example, via the energy consumption or the state of the actuator.

FIG. 10 shows a front view an orthopedic joint device as part of what is called a knee-ankle-foot orthosis (KAFO) in the applied state on a right leg. The upper part 10 has a shell-like fastening element in the form of a thigh shell 11, which is fastened on a thigh. The fastening is carried out with repeatedly releasable fastening elements such as straps, buckles or the like. The lower part 20 is designed correspondingly and has a lower-leg shell 21 for receiving the lower leg. The lower part 20 is also fastened in a repeatedly releasable manner on the leg with straps, buckles or the like. In the illustrated embodiment, the pivot axis 15 is arranged at the height of the natural knee-joint axis, such that the upper part 10 and the lower part 20 can follow the movement during flexion and extension of the leg. Between the upper part 10 and the lower part 20, a first actuator 103 is arranged laterally and a second actuator 105 is arranged medially. The first actuator 103 forms a first joint 101, and the second actuator 105 forms a second joint 102. Alternatively, the first actuator 103 can be arranged on a first joint 101 and the second actuator 105 on a second joint 102. The actuators 103, 105 influence the pivotability of the lower part 20 relative to the upper part 10. The actuators can be designed for this purpose as a motor drive or damper, in particular as a hydraulic damper, pneumatic damper, magnetorheological damper or rotary hydraulics, in order to apply a movement resistance to the flexion movement and/or extension movement. Alternatively, the actuators can also be designed as a releasing and locking device in order to block the flexion movement and/or extension movement if this is desired or required. The first actuator 103 has a first adjustment element 104, and the second actuator 105 has a second adjustment element 106. By way of the adjustment elements, the movement resistance of the associated actuator against a pivoting movement of the lower part 20 relative to the upper part 10 about the pivot axis 15 is adjustable. Furthermore, a sensor 107 is arranged on the upper part 10, which sensor 107 is connected to a control unit 108. For example, the sensor 107 can detect the spatial position, forces, moments, accelerations, speeds, angles and other influencing variables or condition variables of the orthopedic joint device and transmits sensor data to the control unit 108. The sensor 107 can also be arranged within the upper part 10 or at another location of the orthopedic joint device. A plurality of sensors 107 may also be connected to a control unit 108. The control unit 108 is connected to the first adjustment element 104 and the second adjustment element 106 and controls these synchronously in accordance with the sensor data of the sensors.

Arranged below the lower part 20 is a foot part 109, which has a foot shell 110. As an alternative to a foot shell, it is also possible to provide a rail or support. Between the lower part 20 and the foot part 109, a third actuator 114 is arranged laterally and a fourth actuator 116 is arranged medially. The third actuator forms a third joint 112, and the fourth actuator 116 forms a fourth joint 113. The actuators 114, 116 influence the pivotability of the foot part 109 relative to the lower part 20 about the further pivot axis 111. The third actuator 114 has a third adjustment element 115, and the fourth actuator 116 a fourth adjustment element 117. By way of the adjustment elements 115, 117, the movement resistance of the associated actuator against a pivoting movement of the foot part 109 relative to the lower part 20 about the further pivot axis 111 is adjustable. The lower part 20 has a further control unit 118, which is connected to a sensor 107, the third adjustment element 115 and the fourth adjustment element 117. The further control unit 118 controls the third adjustment element 115, and the fourth adjustment element 117 synchronizes according to sensor data of the sensors 107. The sensor setup 107 can be configured differently in the case of several control units 118.

FIG. 11 shows a variant of the embodiment according to FIG. 10, in which only a single control unit 108 and no further control unit 118 is provided. The control unit 108 is arranged on the lower part 20 in this embodiment. In principle, it is also possible to arrange the control unit 108 on the upper part 10 or on the foot part 109. Not only are the first adjustment element 104 and the second adjustment element 106 controlled by the control unit 108, but also the third adjustment element 115 and the fourth adjustment element 117. It is possible that all of the adjustment elements are activated synchronously in accordance with sensor data of the sensors 107. Alternatively, a separate synchronized control can be provided for the adjustment elements of each pivot axis 15, 111. For example, it is conceivable that the first adjustment element 104 and the second adjustment element 106 are controlled synchronously, and that the third adjustment element 115 and the fourth adjustment element 117 are controlled synchronously.

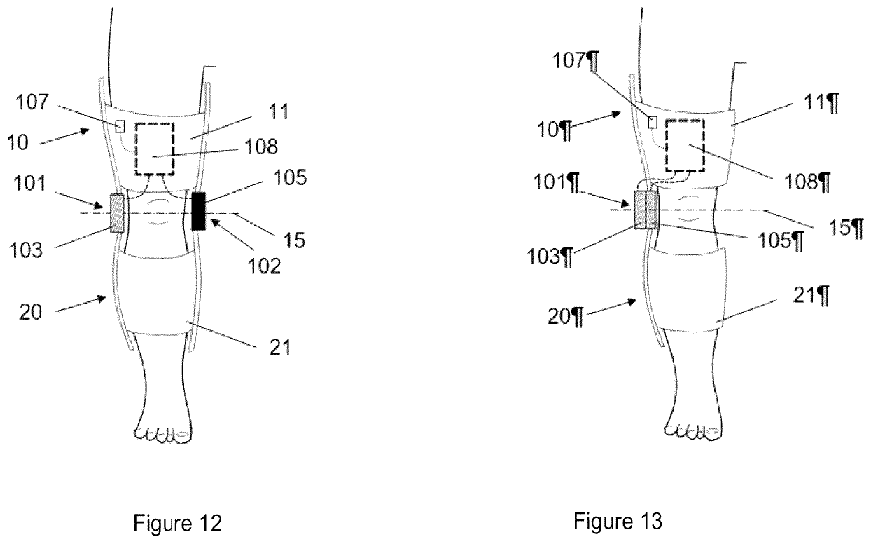

A variant of the orthopedic joint device in the form of a knee orthosis is shown in FIG. 12. The orthopedic joint device consists of an upper part 10 and a lower part 20, the latter being arranged pivotably relative to the upper part 10 about the pivot axis 15. The upper part 10 has a thigh shell 11 for receiving the thigh. The lower part 20 correspondingly has a lower-leg shell 21 for receiving the lower leg. Between the upper part 10 and the lower part 20, a first actuator 103 is arranged laterally and a second actuator 105 is arranged medially. The two actuators 103, 105 act about the pivot axis 15. The first actuator 103 forms a first joint 101, and the second actuator 105 forms a second joint 102. Alternatively, the first actuator 103 is arranged on a first joint 101 and the second actuator 105 on a second joint 102. In this embodiment, the first actuator 103 and the second actuator 105 are based on a different mechanical operating principle. For example, the lateral actuator 103 is designed as a motor drive or as a hydraulic resistance device, in particular as a rotary hydraulics device, while the medial actuator 105 is designed as a releasing and/or locking device. The upper part has a sensor 107 and a control unit 108. The adjustment elements 104 and 106 (not shown) are controlled synchronously by the control unit 108.

FIG. 13 shows a variant of the embodiment shown in FIG. 12, one in which both actuators 103, 105 are arranged laterally on the knee. The first actuator 103 and the second actuator therefore form a common joint or are arranged thereon. On account of the exclusively lateral arrangement of the actuators, there is greater freedom of movement on the medial side. In addition, there is no need for medial rails, and therefore a more lightweight construction can be achieved.

FIG. 14 shows a further embodiment, in which the orthopedic joint device is designed as a leg prosthesis. The upper part 10 has a receptacle 120 for a leg stump, and an attachment means 119, for example in the form of a pyramid adapter. The lower part 20 is arranged pivotably about the pivot axis 15 on the upper part 10. A foot part 109 is arranged distally on the lower part 20 and can be designed as a foot prosthesis. A first actuator 103 and a third actuator 114 are arranged laterally between the upper part 10 and the lower part 20 at the pivot axis 15. A second actuator 105 and a fourth actuator 116 are arranged on the medial side. The lateral actuators 103, 114 form a first joint 101, and the medial actuators 105, 116 form a second joint 102. Furthermore, a sensor 107 is arranged at the pivot axis. Alternatively, the sensor can also be provided elsewhere on the upper part 10 or the lower part 20. For example, the sensor 107 can detect the spatial position, forces, moments, accelerations, speeds, angles and other influencing variables or condition variables of the orthopedic joint device and transmits sensor data to the control unit 108. For capturing this data, the orthopedic joint device can also have further sensors. The actuators 103, 105, 114, 116 each have an adjustment element (not shown). The adjustment elements 104, 106, 115, 117 (not shown) are connected to the control unit 108 and are controlled synchronously by the latter in accordance with sensor data. Through the arrangement of a plurality of actuators on the medial and lateral sides, the orthopedic joint device can be adapted to the body weight of a user within the context of a modular system. With a low body weight of the user, for example, it may be sufficient if only the first actuator 103 and the second actuator 105 are provided. With a higher body weight of the user, a third actuator 114 can be attached to the first actuator 103, and a fourth actuator 116 to the second actuator 105. For securely positioning and fastening the actuators, these can have corresponding form-fit elements, e.g. a dovetail connection or a claw coupling. Alternatively or in addition, a force-fit connection of the actuators can be provided, e.g. by screwing or riveting, and/or a material connection, e.g. by soldering, welding or gluing. In another variant, further actuators are arranged on both the medial side and the lateral side. The electrical connection is ensured by releasable contacts or plugs. In principle, the number of actuator elements can vary as desired, depending on the patient's needs.

FIGS. 15 and 16 show a further variant of the orthopedic joint device in the form of an artificial knee joint, for example an artificial orthotic knee joint with an upper part 10 and a lower part 20, which are mounted pivotably on each other about a pivot axis 15. FIG. 16, in which a cover is removed, shows, in addition to a central bearing 120, the blocking device 30 which can be operated via an actuation element 40 in the form of a lever and an actuator 50. On the upper part 10, an extension stop 5 and a flexion stop 6 are also formed, which define the maximum positions of the upper part 10 relative to the lower part 20. The stops 5, 6 in the illustrated embodiment are fixed; they can also be designed in principle to be adjustable. In the position shown, the orthopedic joint device is in the maximum extended position. The central bearing 120 connects the upper part 10 to the lower part 20 and transmits loads in the axial direction and likewise in the medial direction and lateral direction. A freewheel arranged within the joint device is decoupled from these loads by the central bearing and acts only about the pivot axis 15 or the knee axis. In a movement of the inner ring 31 with respect to the outer ring 32 counter to the blocking direction, the respective blocking elements 35 become tilted, so that they spread and become trapped between the inner ring 31 and the outer ring 32, and locking takes place in the desired blocking direction. In the opposite direction, there is no blocking effect by the blocking device 30, and therefore pivoting is always possible in this direction. For example, extension is always possible, and flexion always blocked, unless the actuator 50 and the actuation element 40 have taken up a corresponding position in order to disengage the blocking elements 35 from the corresponding components. By way of the blocking device 30 and the actuator 50, the respective blocking element 35 or the blocking elements 35 are disengaged from the outer ring 32 or the inner ring 31 via the actuation element 40, so that no locking takes place.

FIG. 17 shows a detailed view of the upper part 10 with the switchable clamping body freewheel. The blocking device has two cages 33, 34, which are arranged between the inner ring 31 and the outer ring 32. The inner cage 33 and the outer cage 34 are mounted rotatably to each other and are each coupled to the upper part 10 and the actuation element 40. If the two cages 33, 34 are rotated relative to each other, the blocking elements 35 rotate, the latter being arranged between the inner ring 31 and the outer ring 32 and engaging with corresponding engagement components of the inner cage 33 and outer cage 34, respectively. During a rotation of the cages relative to each other, the engagement components have the effect that the blocking elements are lifted from the inner ring 31 or the outer ring 32, as a result of which a pivotability of the upper part 10 relative to the lower part 20 is provided in both directions. FIG. 18 shows a sectional view through the blocking device with the switchable clamping body freewheel, from which it will be seen that the blocking elements 35 are lifted from the inner ring 31 by rotation of the inner cage 33 in a clockwise direction. The blocking elements 35 are assigned a prestressing element 37 in the form of a blocking element spring, which prestresses the blocking elements 35 in the direction of the inner ring 31, so that, in the event of no rotation, the blocking elements 35 are automatically reset or pretensioned in the direction of the inner ring 31. Depending on the orientation, it is also possible for them to lift away from the outer ring 32.

FIG. 19 shows a detailed view of the implementation of the circuit and the function with the double cage according to FIGS. 17 and 18. The actuation element 40 is subjected to an actuating force Fa via an actuator not shown. A switching spring 400 can be arranged between the actuator and the actuation element 40. The actuation element 40 is coupled to the outer cage 34 and causes a rotation of the outer cage 34 relative to the inner cage 33. The detailed view on the right shows the applied forces; in the illustrated embodiment, the force Foc through the outer cage acts to the left, while the corresponding counteracting supporting force Fic acts from the inner cage 33. The engagement components are each arranged between the blocking elements 35. The switching spring 400 serves to compensate for deformation-induced movements under load. As an alternative to the actuating force Fa, the cages can also be subjected directly to a moment.

A variant is shown in FIGS. 20 and 21, where FIG. 20 represents a side view of the blocking device with the freewheel. The blocking elements 35 are secured, between the inner ring 31 and the outer ring 32, on the inner ring 31 against displacement along the circumference and, via an outer cage 34, are disengaged from the outer ring 32 if a free pivotability is to be provided in both directions. If the outer ring 32 rotates clockwise with a fixed inner ring 31, there is no blocking effect by the blocking elements 35; in an opposite movement without the activation of the outer ring 34, a rotation is blocked. In FIG. 21, the outer ring 32 is shown as a closed ring with a substantially smooth inner surface and a smooth inner circumference, within which the inner ring 31 with blocking elements 35, the outer cage 34 and the prestressing element 37 are arranged. The prestressing element 37 ensures that the blocking elements 35 bear one the inner circumference of the outer ring 32, provided that the outer cage 34 is not in the release position. Analogously, the functionalities of inner ring 31 and outer ring 32 can also be reversed, i.e. the blocking elements 35 are secured on the outer ring 32 against displacement along the circumference, in which case the inner ring 31 then has to be smooth.

The actuation of the embodiment according to FIGS. 20 and 21 is schematically illustrated in FIG. 22. Here too, a switching force Fa is applied by an actuator, with interposition of the optional switching spring 400, and transmitted to the actuation element 40, which is coupled to the outer cage 34. The blocking elements 35 are mounted in the inner ring 31 in recesses and can be pivoted. If the outer cage 34 is moved to the left in a clockwise direction, a force Foc is applied to the blocking elements 35 by the engagement components of the outer cage 34, so that the blocking elements are lifted from the outer ring. This allows a free movement in both directions.

FIG. 23 shows schematically an embodiment of the support of the blocking elements 35 on the inner ring 31. The blocking element 35 bears on a corresponding recess or bearing location of the inner ring 31 and has a rounded or circular lower contour 351. The lower contour 351 largely corresponds to the shape of the receptacle in the inner ring 31. The rounded shape increases the contact surface between the blocking element 35 and the inner ring 31, as a result of which the surface pressures and thus the contact stresses are reduced. The upper contour 352 has a larger radius than the lower contour 351 and bears on the inner circumference of the outer ring 32. The larger the radius of the upper contour 352, the larger the contact surface with the outer ring 32.

FIG. 24 shows a sectional view of the blocking device with the inner ring 31 and the outer ring 32. The inner ring 31 has recesses in which the blocking elements 35 are arranged with the rounded lower contour 351. Each of the blocking elements 35 is assigned an unlocking pin 45, which is displaceable via the actuation element 40 (not shown). Upon corresponding actuation of the unlocking pin 45 or of the unlocking pins 45, the blocking elements 35 are lifted from the outer ring 32, such that a free mobility of the upper part 10 relative to the lower part 20 is provided in both directions of pivoting. The inner ring 31 is mounted on the outer ring 32 via roller bearings 310 in order to ensure that the components are sufficiently supported on one another during an authorized rotational movement.

In FIG. 25, the blocking elements 35 are mounted as in FIG. 24 and can be disengaged from the outer ring 32 via unlocking pins 45. In addition, prestressing elements 37 are arranged as springs in guides in the inner ring 31 and press the blocking elements 35 against the outer ring 32 and thus ensure contact, hence clamping, upon rotation in the blocking direction. By the unlocking pins 45, the blocking elements 35 can be rotated counter to the force of the prestressing elements 37, as a result of which the outer ring 32 is rotatably released in both directions.

The actuation mechanism of the device according to FIG. 25 is shown in FIG. 26. Here too, the switching force Fa is compensated or limited via the optional switching spring 400 and an elastic flexibility of the actuation element 40 is permitted. The unlocking pins 45 are moved as far as actuation element 40. The effective force Fp is shown in the enlarged view. As soon as the force Fp through the unlocking pin 45 is greater than the prestressing force of the prestressing element 37, the blocking elements 35 are lifted from the outer ring 32, such that the outer ring 32 is freely pivotable in both directions.

FIGS. 27 to 29 show different bearing concepts. In FIG. 27 the inner ring and outer ring are mounted on each other via roller bearings 310, such that an integrated roller bearing is present in the blocking device 30. In FIG. 28, an integrated plain bearing 320 is fastened to the inner ring 31, such that the outer ring 32 is mounted on the plain bearing of the inner ring 31. In FIG. 29, a blocking device with central bearing via a plain bearing 330 is shown on the main axis.

A variant of the invention is shown in FIG. 30, in which the outer ring 32 is slotted and mounted in a manner secure against rotation in the lower part 20. As an alternative to the bearing in the lower part 20, the outer ring 32 can also be arranged or formed in the upper part 10. The outer ring 32 has a slot 324 through which it is possible to adjust the inner circumference of the outer ring 32. The protection against twisting within the lower part 20 is effected, for example, via a recess into which a radial projection of the outer ring 32 is inserted. Alternative fastening devices or fastening options such as welding, soldering, gluing, wedging or similar are also possible. An annular gap 325 is present between the outer circumference of the outer ring 32 and the housing or the bearing location in the lower part 20, such that the outer circumference and thus also the inner circumference of the outer ring 32 can be changed by increasing or decreasing the size of the slot 324. The width of the slot 324 is changed via the prestressing device 327, in which a prestressing spring 326 is prestressed, for example via a prestressing screw 328. Via the prestressing spring 326, it is possible to permit a certain elasticity even in the positioning of the blocking elements 35 in the blocked position. Even in the blocked position, a slight shift of upper part relative to lower part can take placed if a load is applied. The width of the slot 324 is adjustable via the prestressing device 327 and optionally the spring 326.

Thus, even with a closed slot 324, an elasticity of the blocking device 30 counter to the blocking direction can be changed and adjusted via a force that acts in the direction of increasing the size of the slot 324. In particular, through the use of a prestressing spring 326 with a progressive spring characteristic, the stiffness against the blocking device can be adjusted.

Another possibility of changing and adjusting the elasticity counter to the blocking direction can be achieved through a suitable geometry of the blocking elements 35. In the illustrated embodiment shown in FIG. 31, such a blocking element has three elasticity regions α1, α2, α3, which have different radii of curvature and different centers of curvature with respect to their outer contour. The corresponding elasticity regions are applied to the upper contours and lower contours, h_{A} being the initial gap width between the points A on the upper and lower contours, where h_{D} is the final gap width between the points D on the upper and lower contours. The contour of the outer and inner contact surfaces of the blocking element 35 can be selected flexibly.

In particular, the elasticity regions can be realized via the contouring of the outer contact surface, wherein a largely circular contour, as shown in FIG. 23, can be realized on the inner contact surface.

The elasticity of the blocking device 30 is directly related to the expansion of the outer ring 32, divided by the expansion of the outer ring 32 relative to the inner ring 31. The expansion results from the gap width between the inner ring 31 and the outer ring 32. A rotation and expansion of the outer ring 32 results from the rotation of the blocking elements 35. The blocking elements 35 have a blocking element contour which is formed by at least one, advantageously several tangentially overlapping arc sections, preferably circular arc sections. The gap width between the inner ring 31 and the outer ring 32 changes after the rotation of the blocking elements 35, which is coupled in the blocking direction, by the frictional connection, with the rotation of the pivot axis 15. Through the shaping of the blocking elements 35, the elasticity behavior about the pivot axis 15 can be influenced, resulting in at least one elasticity region, in particular several elasticity regions. Through the shaping of the blocking elements 35, it is possible to realize both linear and non-linear, in particular progressive, spring characteristics. According to FIG. 31, three elasticity regions are present. It appears advantageous in the application to initially have a relatively soft elasticity region for an initial knee flexion, in order to allow a sufficient stance flexion at lower moments. In the middle elasticity region, the elasticity could be reduced by the contour geometry in order to provide sufficient rigidity and safety. In the third elasticity region and end region, the contour is for example designed such that the blocking device begins to slip at a predetermined load in order to effect overload protection.

1. An orthopedic joint device, comprising:
an upper part;
a lower part mounted on the upper part so as to be pivotable about a pivot axis; and
a blocking device which blocks a pivoting movement of the upper part relative to the lower part,
wherein the blocking device never blocks in one direction of pivoting and,
wherein in an opposite direction of pivoting, the blocking device is switchable from a release position to a blocking position; and
an actuation element assigned to the blocking device that holds the blocking device in the release position or moves the blocking device to the release position.

2. The orthopedic joint device as claimed in claim 1, wherein the actuation element is assigned an actuator, which holds the actuation element in the release position or moves the actuation element to the release position.

3. The orthopedic joint device as claimed in claim 1 wherein the blocking device has at least one blocking element, which is mounted pivotably or slideably on the upper part and is engageable with a blocking region on the lower part or vice versa.

4. The orthopedic joint device as claimed in claim 3, wherein the at least one blocking element is formed as a blocking bracket, blocking angle, blocking pad, blocking wedge, brake lining, or blocking hook, wherein the at least one blocking element when in a blocking position bears on the blocking region.

5. The orthopedic joint device as claimed in claim 3 wherein the at least one blocking element is in frictional contact with the blocking region.

6. The orthopedic joint device as claimed in claim 3 wherein the at least one blocking element is elastically prestressed in a direction of the blocking region,

7. The orthopedic joint device as claimed in claim 6, wherein the at least one blocking element is mounted on resilient arms.

8. The orthopedic joint device as claimed in claim 7, wherein the resilient arms are assigned an adjustable support.

9. The orthopedic joint device as claimed in claim 1 further comprising an elastic element is mounted to the orthopedic joint device upstream of the blocking device.

10. The orthopedic joint device as claimed in claim 9, wherein a stiffness and/or a prestressing of the elastic element is adjustable.

11. The orthopedic joint device as claimed in claim 10, wherein the elastic element is assigned an adjustable support.

12. The orthopedic joint device as claimed in claim 3, wherein the at least one blocking element is mounted on a coupling element, which is rigidly or elastically connected to the upper part or the lower part.

13. The orthopedic joint device as claimed in claim 1 wherein the blocking device is equipped with an overload protection.

14. The orthopedic joint device as claimed in claim 13, wherein the overload protection comprises a clamping element, whereby a parallel force-fit arises in an event of an overload.

15. The orthopedic joint device as claimed in claim 3, wherein the at least one blocking element is designed or mounted in such that, in an event of an overload, a slip-through occurs.

16. The orthopedic joint device as claimed in claim 1, further comprising a central bearing, wherein the blocking device is guided via the central bearing.

17. The orthopedic joint device as claimed in claim 15, wherein the upper part and/or the lower part are in direct contact with both the central bearing and the blocking device.

18. The orthopedic joint device as claimed in claim 3 wherein the blocking device comprises an outer ring and an inner ring, wherein the at least one blocking element is arranged between the outer ring and the inner ring.

19. The orthopedic joint device as claimed in claim 18, wherein the outer ring is elastic or slotted.

20. The orthopedic joint device as claimed in claim 19, wherein the outer ring has an elasticity that is adjustable by a choice of material and/or a wall thickness and/or a choice of an internal diameter and/or via an axial width of the outer ring.

21. The orthopedic joint device as claimed in claim 19, wherein the outer ring is slotted and is mounted in a manner secure against rotation in a housing.

22. The orthopedic joint device as claimed in claim 19 wherein the outer ring is slotted and is assigned a prestressing device for changing an inner circumference of the outer ring.

23. The orthopedic joint device as claimed in claim 19 wherein a rotational elasticity in a blocking direction is changeable via the outer ring and/or a geometry of the at least one blocking elements.

24. The orthopedic joint device as claimed in claim 23, wherein the rotational elasticity in the blocking direction has a non-linear spring profile.

25. The orthopedic joint device as claimed in claim 1 further comprising:
at least one joint connecting the upper part and the lower part such that the lower part is arranged pivotably about the pivot axis on the upper part, wherein the at least one joint comprises a first actuator and at least a second actuator arranged between the upper part and the lower part;
a first adjustment element and at least a second adjustment element, via which movement resistance of the respectively assigned first actuator and the at least a second actuator against a pivoting movement of the lower part relative to the upper part about the pivot axis is adjustable; and
at least one sensor and a control unit, wherein the control unit is connected to the at least one sensor for transmitting sensor data, and wherein the control unit controls the first adjustment element and the at least a second adjustment element synchronously in accordance with sensor data.

26. The orthopedic joint device as claimed in claim 25, wherein the first actuator and the at least a second actuator are controlled synchronously by the control unit.
